(19) United States
(12) Patent Application Publication
(10) Pub. No.: US 2019/0029542 A1
(43) Pub. Date: Jan. 31, 2019

(54) FINGER CUFF

(71) Applicant: Edwards Lifesciences Corporation, Irvine, CA (US)

(72) Inventors: Peiyuan Li, Amsterdam (NL); Jeroen Van Goudoever, Amstelveen (NL)

(21) Appl. No.: 16/045,378

(22) Filed: Jul. 25, 2018

Related U.S. Application Data
(60) Provisional application No. 62/539,317, filed on Jul. 31, 2017.

(51) Int. Cl. A61B 5/022; A61B 5/0225; A61B 5/00

(52) U.S. Cl. CPC A61B 5/02241; A61B 5/02255; A61B 2562/227; A61B 5/6826; A61B 5/6831

(57) ABSTRACT

Disclosed is a finger cuff that is attachable to a patient's finger to be used in measuring the patient's blood pressure. The finger cuff includes a first side and a second side. The first side is removably attached to the second side to wrap the finger cuff around the patient's finger. The finger cuff further includes a signal source and signal detector pair and a bladder. The bladder includes a pair of openings and is mountable within the finger cuff such that the pair of openings surround the signal source and signal detector pair, respectively. The patient's finger surrounded in the finger cuff abuts against the bladder such that the bladder and the signal source and signal detector pair are used in measuring the patient's blood pressure. Further, the bladder and signal source and signal detector pair are coupled to a cable through a connector assembly. The cable provides pneumatic pressure to the bladder and receives electrical signals from the signal source and signal detector pair.

### CROSS-REFERENCE TO RELATED APPLICATIONS

This application claims the benefit of U.S. Provisional Patent Application No. 62/539,317, filed Jul. 31, 2017, which is incorporated herein by reference.

### BACKGROUND

### Field

Embodiments of the invention relate generally to finger cuffs.

### Relevant Background

Volume clamping is a technique for non-invasively measuring blood pressure in which pressure is applied to a patient's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the patient's blood pressure.

For a proper functioning of the volume clamp based blood pressure measurement, the design and application of the finger sensor are critical. The bladder inside the sensor should be small enough not to influence the quick pressure changes that are needed for optimal control of the arterial volume, and the bladder should be large enough to properly transfer the cuff pressure to the underlying artery. Due to the use of a photo-plethysmograph, the cuff orientation is also important to warrant that the arterial diameter changes under the cuff are effectively measured. Compared to the presently used wrap-around and ring type of sensors, the embodiments disclosed here focus on easy and robust application, which are less sensitive to cuff application errors and motion.

### SUMMARY

Embodiments of the invention may relate to a finger cuff that is attachable to a patient's finger to be used in measuring the patient's blood pressure by a blood pressure measurement system. The finger cuff includes a first side and a second side. The first side is removably attached to the second side to wrap the finger cuff around the patient's finger. The finger cuff further includes a signal source and signal detector pair and a bladder. The bladder includes a pair of openings and is mountable within the finger cuff such that the pair of openings surround the signal source and signal detector pair, respectively. The patient's finger surrounded in the finger cuff abuts against the bladder such that the bladder and the signal source and signal detector pair are used in measuring the patient's blood pressure by the blood pressure measurement system. Further, the bladder and signal source and signal detector pair are coupled to a cable through a connector assembly. The cable provides pneumatic pressure to the bladder and receives electrical signals from the signal source and signal detector pair.

In one embodiment, a finger cuff may be connectable to a patient's finger to be used in measuring the patient's blood pressure by a blood pressure measurement system, in which the finger cuff comprises: a top shell portion and a bottom shell portion forming a finger cavity of the finger cuff to be placed around the patient's finger, the bottom shell portion including a signal source and signal detector pair and a bladder. The bladder may include a pair of openings, in which the bladder is mountable within the bottom shell portion such that the pair of openings surround the signal source and signal detector pair, respectively, wherein the patient's finger received and surrounded in the finger cavity abuts against the bladder such that the bladder and the signal source and signal detector pair are used in measuring the patient's blood pressure by the blood pressure measurement system.

In one embodiment, a finger cuff is connectable to a patient's finger to be used in measuring the patient's blood pressure by a blood pressure measurement system. The finger cuff may comprise: a shell having a first slot and a second slot on first and second opposing ends of the shell, the shell forming a finger cavity to be placed around the patient's finger; a flexible material having a first side and a second side, wherein the first side and the second side are respectively inserted through the first slot and the second slot; and a bladder assembly insertable into the shell. The bladder assembly may comprise a shell portion having a signal source and signal detector pair and a bladder including a pair of openings. The bladder may be mountable within the shell portion such that the pair of openings surround the signal source and signal detector pair, respectively, wherein the patient's finger received and surrounded in the finger cavity abuts against the bladder such that the bladder and the signal source and signal detector pair are used in measuring the patient's blood pressure by the blood pressure measurement system.

In one embodiment, a finger cuff is connectable to a patient's finger to be used in measuring the patient's blood pressure by a blood pressure measurement system. The finger cuff may comprise: a fixed shell including a first slot and a second slot; and

a wrapping portion within the fixed shell. The wrapping portion may include: a first side and a second side respectively inserted through the first slot and the second slot forming a finger cavity of the wrapping portion to be placed around the patient's finger; a signal source and signal detector pair; and a bladder including a pair of openings. The bladder is mountable within the wrapping portion such that the pair of openings surround the signal source and signal detector pair, respectively, wherein, the patient's finger surrounded in the finger cavity abuts against the bladder such that the bladder and the signal source and signal detector pair are used in measuring the patient's blood pressure by the blood pressure measurement system.

### BRIEF DESCRIPTION OF THE DRAWINGS

FIG. 1 is a diagram of an example of a blood pressure measurement system according to one embodiment.

FIGS. 2A-2C are views of a finger cuff according to embodiments of the invention.

FIGS. 3A-3D are views of a finger cuff and a connector assembly according to embodiments of the invention.

FIGS. 4A-4D are views of a finger and a snap connector assembly according to embodiments of the invention.

FIGS. 5A-5B illustrate a label of a finger cuff according to embodiments of the invention.

FIG. 6 illustrates several features associated with a finger cuff according to embodiments of the invention.

FIGS. 7A-7D are views of a shell finger cuff with a rotatable knob according to embodiments of the invention.

FIGS. 8A-8F are views of a finger cuff with a bladder assembly according to embodiments of the invention.

FIGS. 9A-9E are views of a finger cuff with a pull member according to embodiments of the invention.

### DETAILED DESCRIPTION

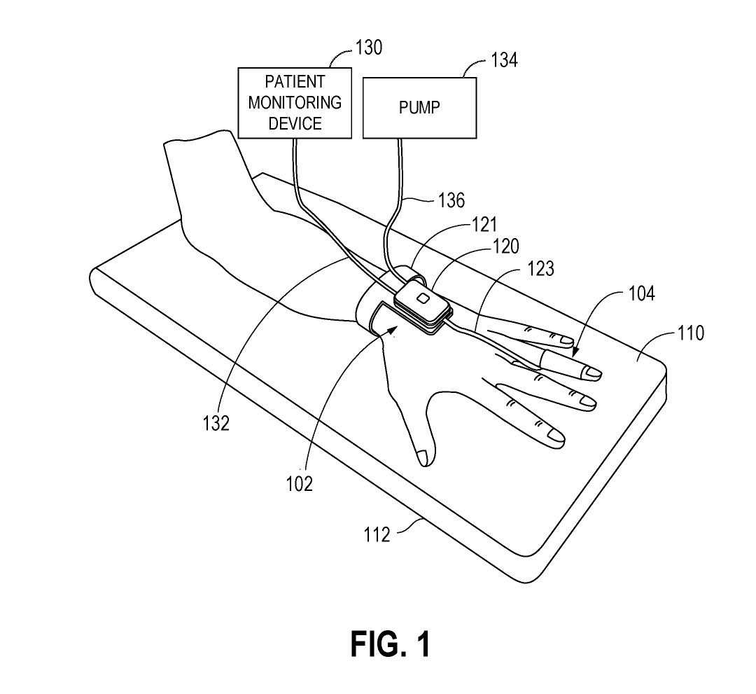

With reference to FIG. 1, which illustrates an example of a blood pressure measurement system according to one embodiment, a blood pressure measurement system 102 that includes a finger cuff 104 that may be attached to a patient's finger and a blood pressure measurement controller 120, which may be attached to the patient's body (e.g., a patient's wrist or hand) is shown. The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuity. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure senor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method. Further, any sort of suitable finger cuff 104 including an LED-PD pair and a bladder may be utilized to implement the volume clamping method. For example, finger cuff 104 may include a suitable wrapping portion that wraps around the patient's finger (e.g., being of flexible material with a Velcro clamping system) that can be connected around the patient's finger and that includes an LED-PD pair and a bladder to aid in measuring the patient's blood pressure by the blood pressure measurement system utilizing the volume clamping method. It should be appreciated that the finger cuffs to be hereafter described may be utilized to measure blood pressure utilizing a blood pressure measurement system utilizing the volume clamp method (as well as other physiological factors). It should be appreciated that the finger cuffs to be hereafter described may also be utilized to measure other physiological and hemodynamic parameters, such as: stroke volume; stroke volume variation; cardiac output; systemic vascular resistance; as well as various types of other parameters.

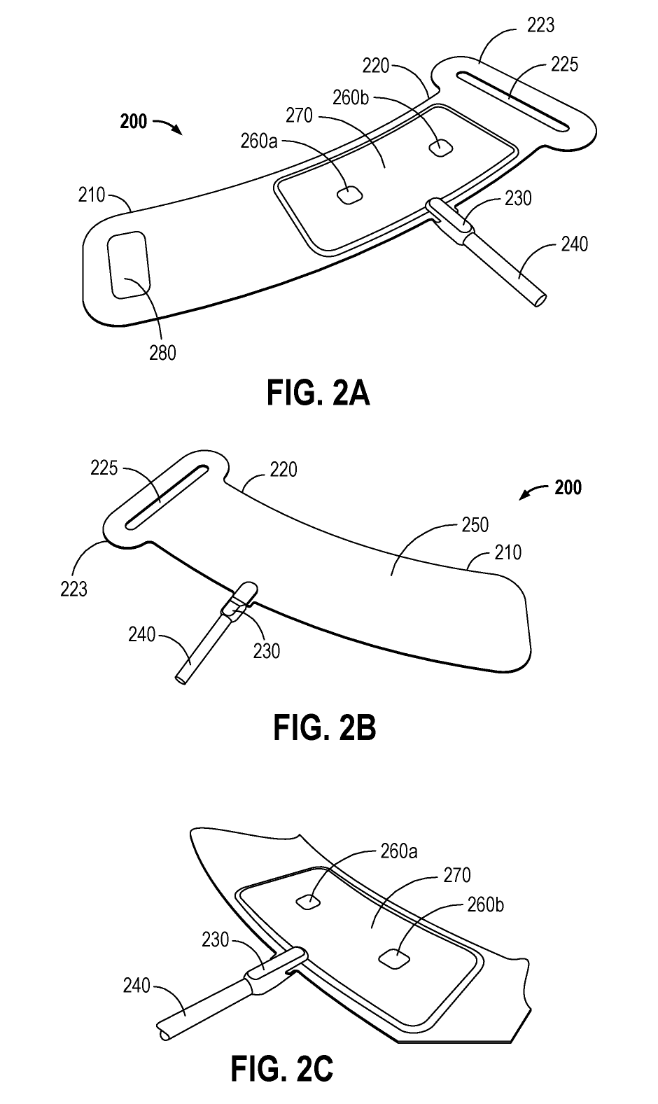

With reference to FIGS. 2A-2C, embodiments of the invention related to a finger cuff 200 will be particularly described. In some embodiments, the finger cuff 200 may be the finger cuff 104, as previously described in FIG. 1. As shown, finger cuff 200 may wrap around a patient's finger. The finger cuff 200 may be formed from a flexible material with a suitable clamping system (e.g., a Velcro system or any suitable releasable adhesive). As further shown in FIGS. 2A-2C, the finger cuff 200 may include a first side 210 and a second side 220. In one embodiment, the second side 220 may include an elongated portion 223 having a slot 225. For attachment purposes to the patient's finger, the first side 210 may be pulled towards the second side 220, for example by a healthcare provider, and inserted through the slot 225, in order to wrap the finger cuff 200 around the patient's finger. As shown in FIG. 2A, in one embodiment, the first side 210 on the interior may include a first connecting portion 280 (e.g., a Velcro type portion) that connects with a second connecting portion (not shown), e.g., a Velcro type portion, on the exterior of the second side 220 of the finger cuff 200. In another embodiment, the first connecting portion 280 may include removable or reusable adhesive material that can be removably attached to the exterior surface of the second side 220 of the finger cuff 200. It should be appreciated that these are just some examples of an attachment mechanism and that any suitable attachment mechanism may be utilized. Further, finger cuff 200 may include a bladder 270 and a signal source and signal detector pair 260a-b mounted on the interior of the finger cuff 200. It should be appreciated the signal source and signal detector pair 260a-b may be an LED-PD pair 260a-b, as is commonly utilized, but may be any suitable signal source and signal detector pair that can be utilized to achieve the intended functions. For ease of reference, an LED-PD pair will be hereafter referred to in all embodiments, but it should be appreciated that any suitable signal source and signal detector pair may be utilized. In one embodiment, the bladder 270 may include a pair of openings that surround the LED-PD pair 260a-b. The bladder 270 and LED-PD pair 260a-b may couple to tube or cable 240 through a fixed connector 230, which may be attached to finger cuff 200, to provide pneumatic pressure to the bladder 270, and to provide power to and receive electrical signals/data from the LED-PD pair 260a-b. It should be appreciated that the bladder 270 and the LED-PD pair 260a-b may be used in measuring the patient's blood pressure by the blood pressure measurement system utilizing the volume clamping method.

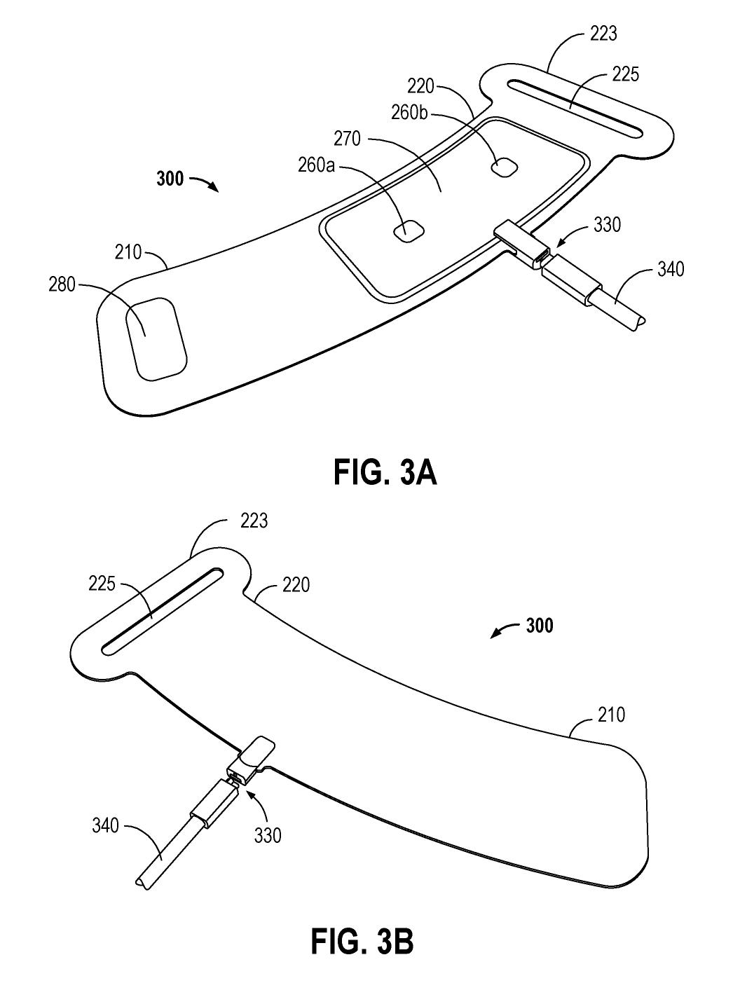

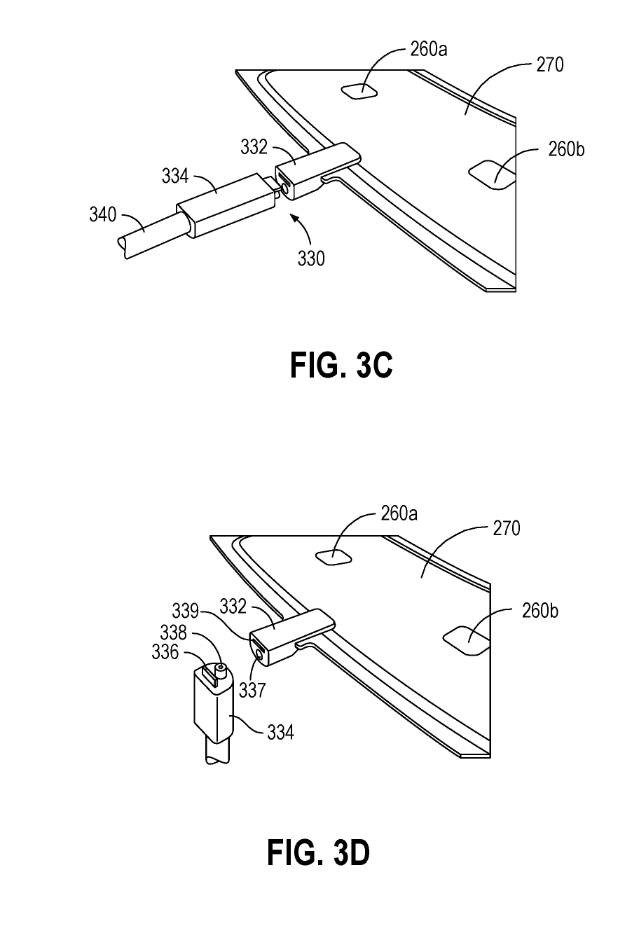

With additional reference to FIGS. 3A-3D, embodiments of the invention related to a finger cuff 300 will be particularly described. In some embodiments, the finger cuff 300 may be the finger cuff 104, as previously described in FIG. 1. The finger cuff 300 may include components that are similar or same as components previously described with respect to finger cuff 200 of FIGS. 2A-2C, such as first side 210, second side 220 that includes elongated portion 223 having slot 225, first connecting portion 280, bladder 270, and LED-PD pair 260a-b. Therefore, the same attachment procedure to the patient's finger will not be repeated.

Further, in FIGS. 3A-3D, the bladder 270 and LED-PD pair 260a-b are coupled to tube or cable 340 through a connector assembly 330. Referring to FIGS. 3C-3D, the connector assembly 330 may include a plug connector 334 and a receptacle connector 332 that are shown in a non-plugged state. The receptacle connector 332, which may have one end attached to finger cuff 300, may be connected or coupled to bladder 270 and LED-PD pair 260a-b, while the plug connector 334 may have one end connected to tube 340. As further shown in FIGS. 3C-3D, the plug connector 334 may include another end having a first plugging member 336 and a second plugging member 338. The first plugging member 336 may include a group of electrical contacts (not shown) and the second plugging member 338 may include a cavity to pass pneumatic pressure to bladder 270. Still referring to FIGS. 3C-3D, the receptacle connector 332 may include another end having a first socket 337 and a second socket 339. By inserting the plug connector 334 into the receptacle connector 332, the group of electrical contacts on the first plugging member 336 may engage a corresponding group of electrical contacts within the second socket 339 such that power may be provided to the LED-PD pair 260a-b and electrical signals (e.g., pleth signal) may be received from the LED-PD pair 260a-b. Further, by inserting the plug connector 334 into the receptacle connector 332, the second plugging member 338 (which may be of cylindrical shape) may be inserted into the first socket 337 such that pneumatic pressure may pass through tube 340 and connector assembly 330 to bladder 270.

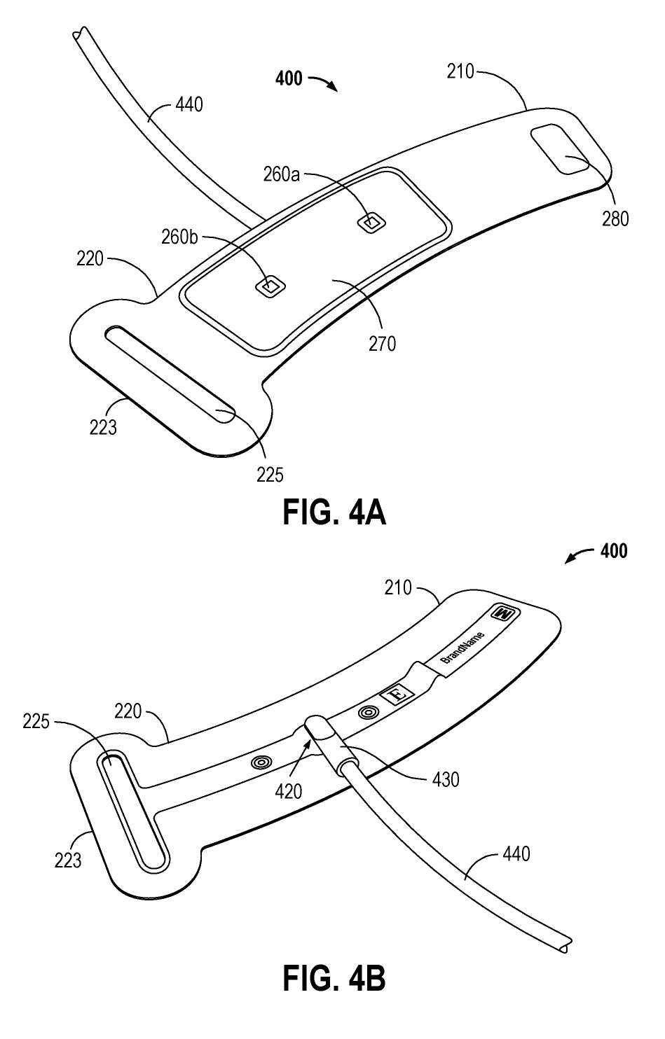

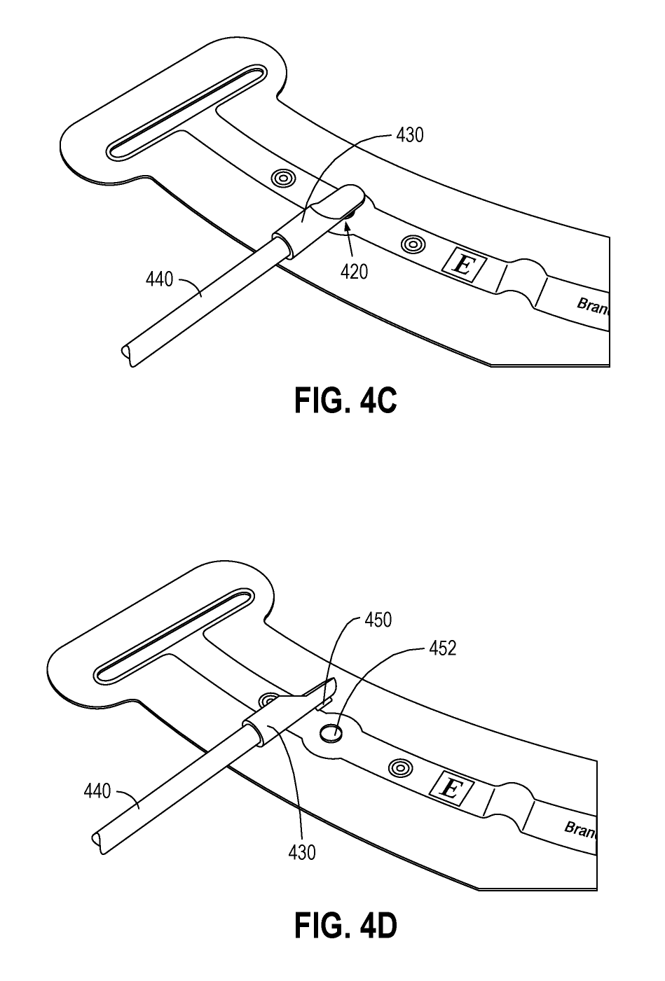

With additional reference to FIGS. 4A-4D, embodiments of the invention related to finger cuff 400 will be particularly described. In some embodiments, the finger cuff 400 may be the finger cuff 104, as previously described in FIG. 1. As with finger cuff 300 of FIGS. 3A-3D, the finger cuff 400 may include components that are similar or same as components previously described with respect to finger cuff 200 of FIGS. 2A-2C, such as first side 210, second side 220 that includes elongated portion 223 having slot 225, first connecting portion 280, bladder 270, and LED-PD pair 260a-b. Therefore, the same attachment procedure to the patient's finger will not be repeated.

Further, in FIGS. 4A-4D, the bladder 270 and LED-PD pair 260a-b are coupled to tube or cable 440 through a snap connector assembly 420. The snap connector assembly 420 may include a snap member 430 connected to tube 440 and having a loop 450 extending from a surface of the snap member 430 and a mating loop 452 attached to the exterior of finger cuff 400. As shown in FIG. 4D, the loop 450 and mating loop 452 are in a disconnected state. By snapping the snapping member 430 into the mating loop 452 (as shown in FIGS. 4B-4C), the loop 450 may be secured to the mating loop 452. Accordingly, pneumatic pressure may be provided to bladder 270 through tube 440 and snap connector assembly 420. Further, power may be provided to the LED-PD pair 260a-b and electrical signals (e.g., pleth signal) may be received from the LED-PD pair 260a-b through tube 440 and snap connector assembly 420. It should be appreciated that the bladder and the LED-PD pair of finger cuffs 104, 200, 300, and 400, may be used in measuring the patient's blood pressure by the blood pressure measurement system utilizing the volume clamping method.

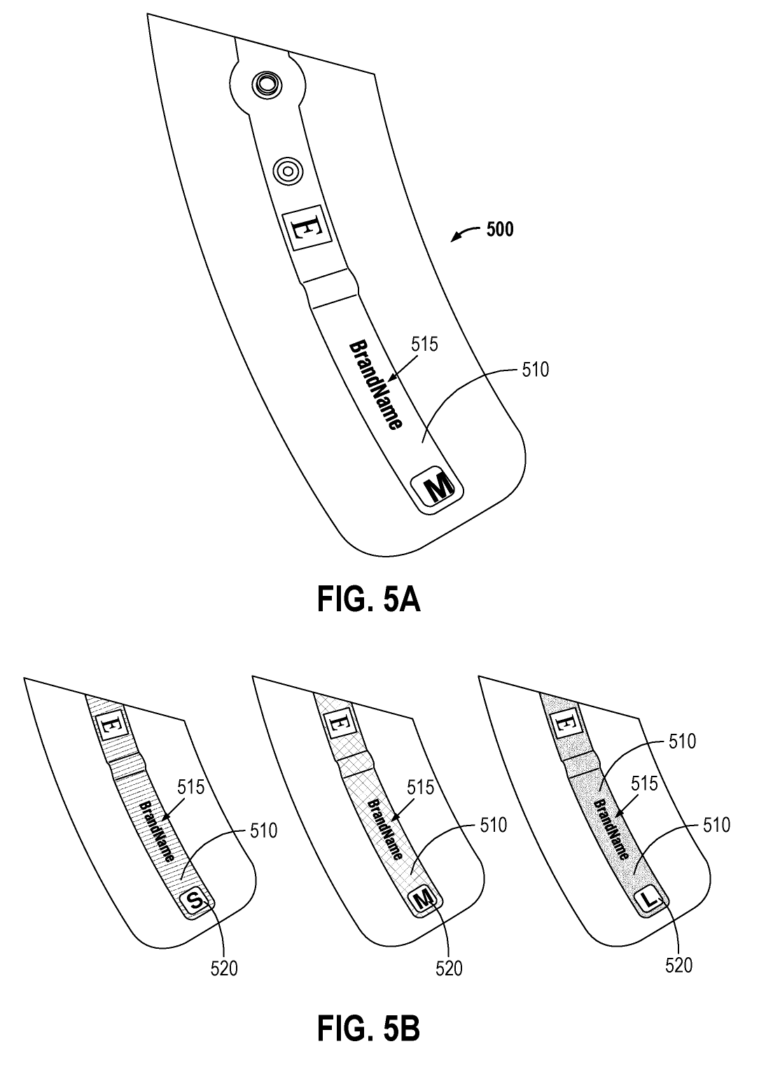

With additional reference to FIGS. 5A-5B, embodiments of the invention related to a label 510 of a finger cuff 500 will be particularly described. In some embodiments, finger cuff 500 may be any of finger cuffs 104, 200, 300 and 400 as previously described. Referring to FIGS. 5A-5B, the exterior of finger cuff 500 may include the label 510 (e.g., a printed label) that displays information pertaining to the finger cuff 500. The label 510 may include a name brand 515, such as CLEARSIGHT from Edwards Lifesciences Corporation, and a letter or character or symbol portion 520 indicating the size of finger cuff 500. For example, the character portion 520 may include a letter “S” indicating a small-sized finger cuff 500, a letter “M” indicating a medium-sized finger cuff 500, and a letter “L” indicating a large-sized finger cuff 500. Other examples may include “XS” indicating extra small or “XL” indicating extra-large. Further, the label 510 and the letter may be of different colors (e.g., green, blue, and orange) to also indicate the size of finger cuff 500. For instance, a green color label 510 and green color letter may correlate to a small-sized finger cuff 500, a blue color label 510 and blue color letter may correlate to a medium-sized finger cuff 500, and an orange color label 510 and orange color letter may correlate to a large-sized finger cuff 500.

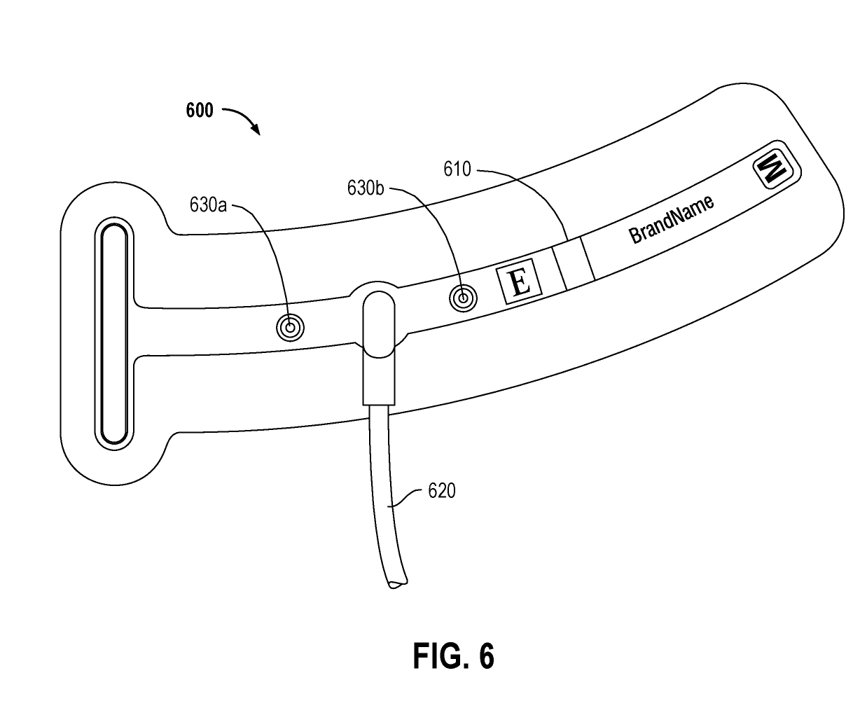

With additional reference to FIG. 6, various features associated with finger cuff 600 will be particularly described. In some embodiments, finger cuff 600 may be any of finger cuffs 104, 200, 300 and 400 as previously described. In FIG. 6, the exterior of the finger cuff 600 may include a loop 610 for attaching a sensor, such as, a heart reference sensor (HRS), to the finger cuff 600. The exterior of the finger cuff 600 may further include sensor symbols 630a-b (which may resemble a shooting target) indicating the positioning of an LED-PD pair (e.g., LED-PD pair 260a-b as previously described) on the interior of the finger cuff 600. That is, the positioning of the sensor symbols 630a-b on the exterior of the finger cuff 600 may correspond to the positioning of the LED-PD pair on the interior of the finger cuff 600. Still referring to FIG. 6, tube or cable 620 (which may be any of tubes/cables 123, 240, 340, 440 as previously described) connected or coupled to the exterior of the finger cuff 600 may be of a particular color such that tube 620 would stand out against cabling associated with various devices.

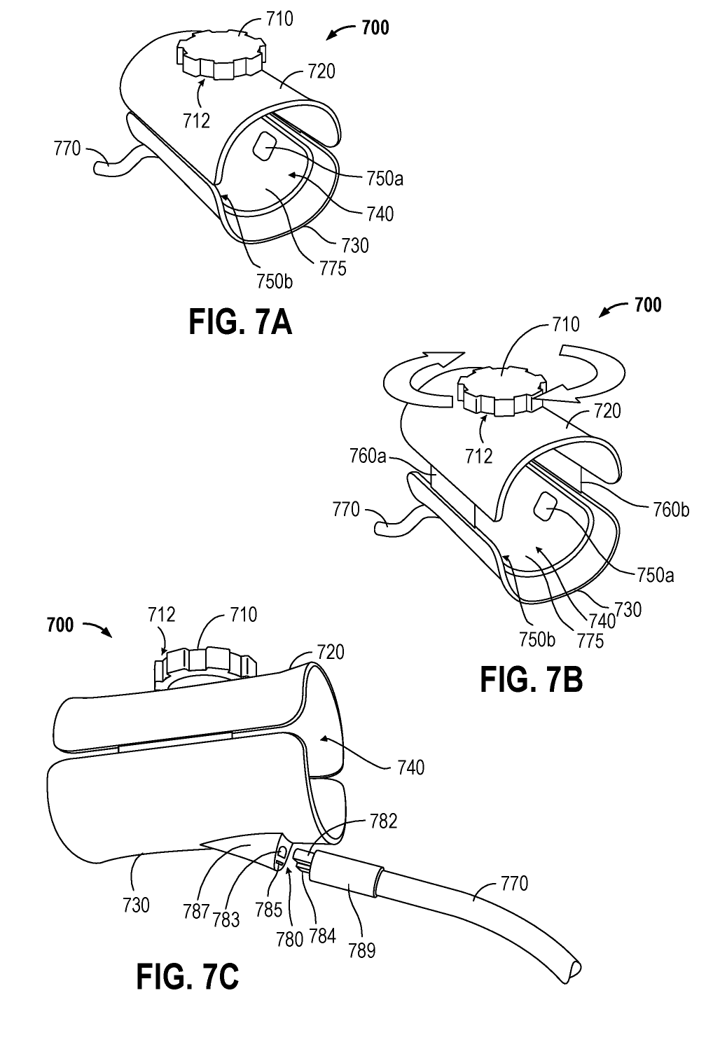

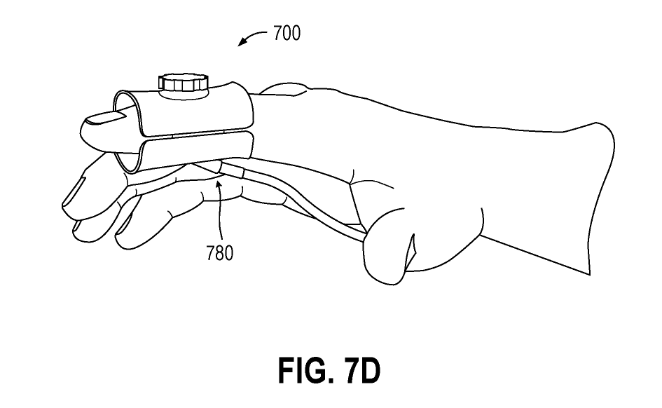

With additional reference to FIGS. 7A-7D, embodiments of the invention related to a shell finger cuff 700 to reduce finger movement will be described. In some embodiments, the finger cuff 700 may be the finger cuff 104, as previously described in FIG. 1. As described with respect to finger cuff 104, finger cuff 700 may be connectable to a patient's finger to be used in measuring the patient's blood pressure by the previously described blood pressure measurement system 102 utilizing the volume clamping method. As can be seen in FIGS. 7A-7D, finger cuff 700 may include a top shell portion 720, a bottom shell portion 730, and opposing adjustable sidewalls 760a-b that extend from the top shell portion 720 to the bottom shell portion 730.

In one embodiment, sidewall 760a may extend from a first end of top shell portion 720 to a first end of bottom shell portion 730. Similarly, sidewall 760b may extend from a second end of top shell portion 720 to a second end of bottom shell portion 730. In one embodiment, the top shell portion 720 and bottom shell portion 730 may be convex in shape and of extended length. In one embodiment, the sidewalls 760a-b may be of flexible material to provide some flexibility in addition to the rigidity of the top shell portion 720 and bottom shell portion 730. Together, top shell portion 720, bottom shell portion 730, and sidewalls 760a-b may form the shell finger cuff 700 that may be approximately cylindrically-shaped and may have an adjustable finger cavity 740 that is approximately oval-shaped for insertion of the patient's finger into the finger cuff 700, for example by the healthcare provider. As can be seen in FIG. 7D, the interior of top shell portion 720 abuts against the topside of the patient's finger, while the bladder 775 on the interior of bottom shell portion 730 abuts against the underside of the patient's finger. It should be appreciated that the top shell portion 720 and bottom shell portion 730 may be formed by any suitable sort of material: plastic, polyvinyl chloride (PVC), metallic material, etc., or combinations thereof, to provide suitable rigidity as well as desired flexibility.

Referring to FIGS. 7A-7B, an LED-PD pair 750a-b and bladder 775 may be mounted on the interior of bottom shell portion 730 and within the finger cavity 740. In one embodiment, the bladder 775 may include a pair of openings that surround the LED-PD pair 750a-b. The exterior of the top shell portion 720 may be coupled or connected to a rotatable knob 710 that adjusts the length of the adjustable sidewalls 760a-b to effectively fit the patient's finger into the finger cuff 700. For example, in one embodiment, the healthcare provider may turn the rotatable knob 710 counter-clockwise to lengthen the sidewalls 760a-b, thereby pushing the bottom shell portion 730 away from the top shell portion 720 in order to create a suitable finger cavity 740 for guidance of the patient's finger into the finger cavity 740. After the patient's finger is inserted, the healthcare provider may turn the rotatable knob 710 clockwise to shorten the sidewalls 760a-b, thereby pulling the bottom shell portion 730 towards the top shell portion 720 in order to tighten the finger cuff 700 around the patient's finger. This would decrease the size of finger cavity 740 to effectively fit the finger cuff 700 around the patient's finger. In another embodiment, the turn direction of the rotatable knob 710 may operate in the reverse such that turning the rotatable knob 710 clockwise would cause the bottom shell portion 730 to move away from the top shell portion 720, and turning the rotatable knob 710 counter-clockwise would pull the bottom shell portion 730 towards the top shell portion 720 in order to tighten the finger cuff 700 around the patient's finger. Accordingly, due to the solid nature of the finger cuff 700, the patient's possible finger movement is significantly reduced thereby reducing noise and increasing the accuracy of the blood pressure measurement (utilizing the volume clamping method). In one embodiment, the rotatable knob 710 may include several grooves, such as groove 712, around the rotatable knob 710 to facilitate the gripping of the rotatable knob 710 by a user (e.g., a healthcare provider). Also, in one embodiment, features may be included to prevent over-tightening by the rotatable knob 710, so the cuff will be applied with the right snugness

Still referring to FIGS. 7A-7D, finger cuff 700 (the bottom shell portion 730 in particular) may be coupled to tube or cable 770 through a connector assembly 780. In one embodiment, the connector assembly 780 may be similar or the same as connector assembly 330 of FIG. 3. Accordingly, as shown in FIGS. 7A-7D, the connector assembly 780 may include a plug connector 789 and a receptacle connector 787 that are represented in a non-plugged state. The receptacle connector 787, which may be attached to the exterior of bottom shell portion 730, may be connected or coupled to bladder 775 and LED-PD pair 750a-b, while the plug connector 789 may be connected to tube 770. As further shown in FIGS. 7C-7D, the plug connector 789 may include a first plugging member 784 and a second plugging member 782. The first plugging member 784 may include a group of electrical contacts (not shown) and the second plugging member 782 may include a cavity to pass pneumatic pressure to bladder 775. Still referring to FIGS. 7C-7D, the receptacle connector 787 may include a first socket 783 and a second socket 785. By inserting the plug connector 789 into the receptacle connector 787, the group of electrical contacts on the first plugging member 784 may engage a corresponding group of electrical contacts within the second socket 785 such that power may be provided to the LED-PD pair 750a-b and electrical signals (e.g., pleth signal) may be received from the LED-PD pair 750a-b. Further, by inserting the plug connector 789 into the receptacle connector 787, the second plugging member 782 (which may be of cylindrical shape) may be inserted into the first socket 783 such that pneumatic pressure may pass through tube 770 and connector assembly 780 to bladder 775.

In some embodiments, the finger cuff 700 is useable with patients who are awake (e.g., not sedated) in a wide variety of different areas (e.g., in the emergency room, a standard hospital, etc.). Also, by utilizing the shell finger cuff 700 with finger cavity 740 that completely surrounds the patient's finger and guides the finger into the finger cuff 700, the patient's finger may be secured with a snug fit such that orientation and rotation errors due to the movement of the finger are avoided, and more accurate blood pressure measurements can be taken by the volume clamp method of the blood pressure measurement system 102. In particular, with the patient's finger received in the finger cavity 740 of the shell finger cuff 700 abutting against the bladder 775, the bladder 775 (being provided pneumatic pressure through the tube 770) and the LED-PD pair 750a-b may be more accurately utilized in measuring the patient's blood pressure by the volume clamp method of the blood pressure measurement system 102.

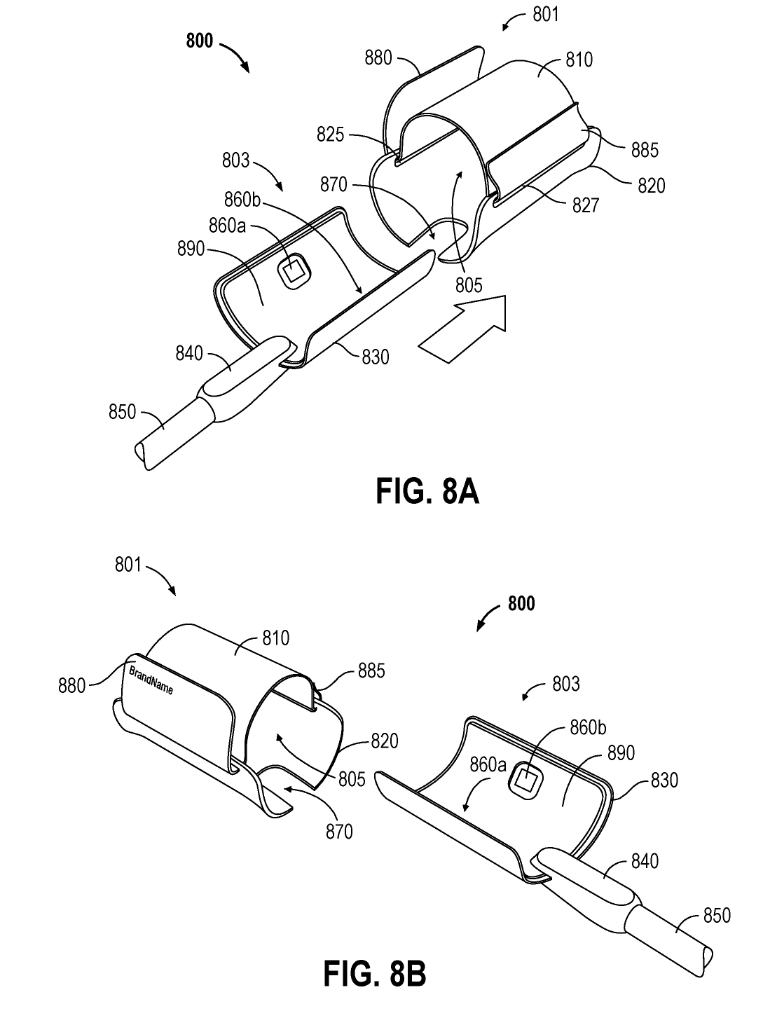

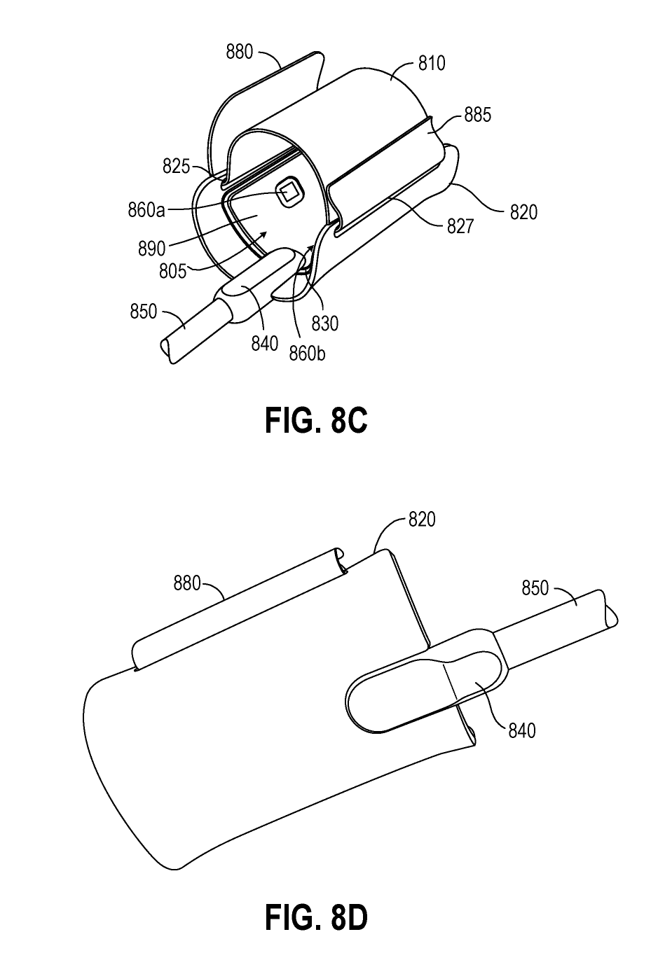

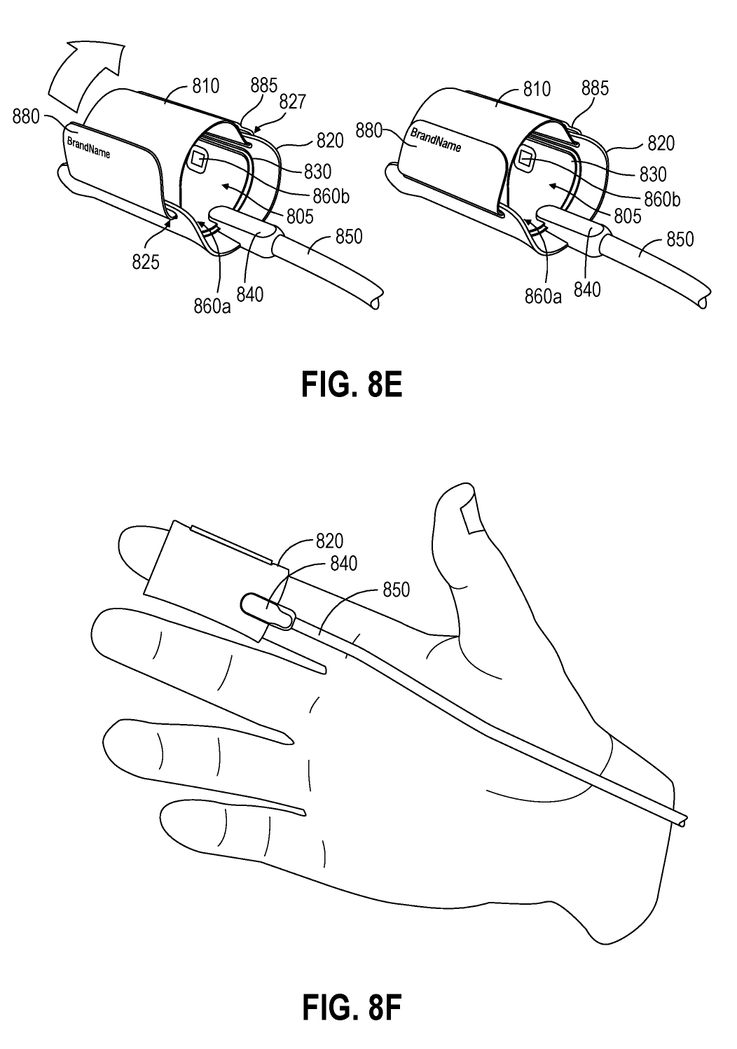

With additional reference to FIGS. 8A-8F, embodiments of the invention related to finger cuff 800 will be particularly described. In some embodiments, the finger cuff 800 may be the finger cuff 104, as previously described in FIG. 1. As shown, finger cuff 800 may include a finger cuff assembly 801 and a bladder assembly 803. The finger cuff assembly 801 may include a flexible material 810 having a first side 880 and a second side 885, and a shell 820 having first slot 825 and second slot 827 on opposing ends of the shell 820, respectively. The shell 820 may be convex in shape and may from a finger cavity 805 and may further include a shell cavity 870 that is located at a convex edge of the shell 820. For attachment purposes to the patient's finger, the first side 880 may be inserted through the first slot 825 and the second side 885 may be inserted through the second slot 827 in order to aid in forming the finger cavity 805 (which may be approximately oval shaped) that helps to guide the patient's finger in, with the flexible material 810 wrapping around the patient's finger. Subsequently, in one embodiment, the first side 880 on the interior may be removably attached to a first outward-facing surface of the flexible material 810 (e.g., using a Velcro clamping system), while the second side 885 on the interior may be permanently fixed to a second outward-facing surface of the flexible material 810. In another embodiment, the second side 885 on the interior may be removably attached to the second outward-facing surface of the flexible material 810 (e.g., using a Velcro clamping system).

With reference to FIGS. 8A-8B, the bladder assembly 803 may include a shell portion 830 (which may be convex in shape), a fixed connector 840, and a tube or cable 850. The shell portion 830 may be coupled to the tube 850 through fixed connector 840, with the fixed connector 840 connecting to both the shell portion 830 and tube 850. Looking at FIGS. 8A-8B, a bladder 890 and a LED-PD pair 860a-b may be mounted on the interior of the shell portion 830. In one embodiment, the bladder 890 may include a pair of openings that surround the LED-PD pair 860a-b. The bladder 890 and LED-PD pair 860a-b may be coupled to tube 850 through the fixed connector 840 to provide pneumatic pressure to bladder 890 and provide power to and receive electrical signals (e.g., pleth signal) from the LED-PD pair 860a-b. It should be appreciated that the fixed connector 840 is just one example of coupling the bladder 890 and LED-PD pair 860a-b to tube 850, and that any suitable connectors or connecting system may be utilized, such as the connector assembly 330 and snap connector assembly 420 as previously described.

As shown in FIGS. 8A-8F, the bladder assembly 803 may be inserted into the finger cuff assembly 801 to form the finger cuff 800, and to cause the bladder 890 and LED-PD pair 860a-b to be positioned within finger cavity 805. In particular, the shell portion 830 (along with the fixed connector 840) may be inserted into the shell 820 until a portion of the fixed connector 840 is fixed or secured into the shell cavity 870. In one embodiment, the exterior of the shell portion 830 may abut the interior of the shell 820. In another embodiment, the exterior of the shell portion 830 may not abut the interior of the shell 820, thereby creating a gap between the exterior of the shell portion 830 and the interior of the shell 820. It should be appreciated that the shell 820 and shell portion 830 of finger cuff 800 may be formed by any suitable sort of material: plastic, polyvinyl chloride (PVC), metallic material, etc., or combinations thereof, to provide suitable rigidity as well as desired flexibility.

In operation, in one embodiment, the healthcare provider may insert the bladder assembly 803 into the finger cuff assembly 801 to form the finger cuff 800. Subsequently, the healthcare provide may guide the patient's finger through the finger cavity 805 such that the bladder 890 would abut the underside of the patient's finger. In another embodiment, the healthcare provider may instead guide the patient's finger through the finger cavity 805 and subsequently, insert the bladder assembly 803 underneath the patient's finger and into the finger cuff assembly 801 such that the bladder 890 would abut the underside of the patient's finger. Subsequently, in one embodiment, the healthcare provider may pull the first side 880 outward, while the second side 885 being fixed to the second outward-facing surface of the flexible material 810, and secure the first side 880 to the first outward-facing surface of the flexible material 810 in order to tighten the flexible material 810, and ultimately the finger cuff 800, around the patient's finger. In another embodiment, the healthcare provider may pull both the first side 880 and the second side 885 outward, and secure the first side 880 to the first outward-facing surface of the flexible material 810 and the second side 885 to the second outward-facing surface of the flexible material 810. It should be appreciated that the bladder 890 and the LED-PD pair 860a-b may be used in measuring the patient's blood pressure by the blood pressure measurement system utilizing the volume clamping method.

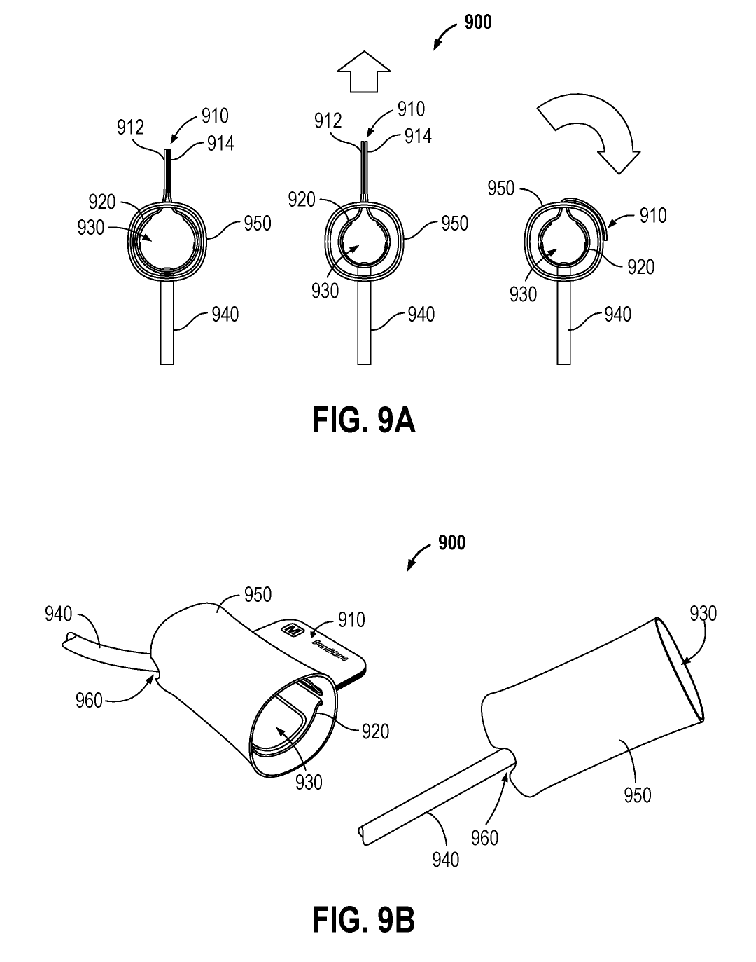

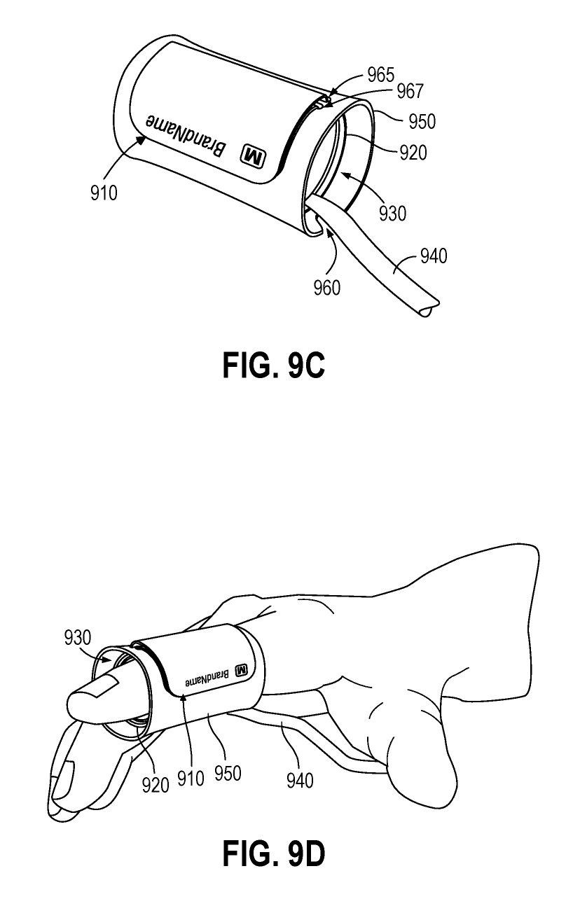

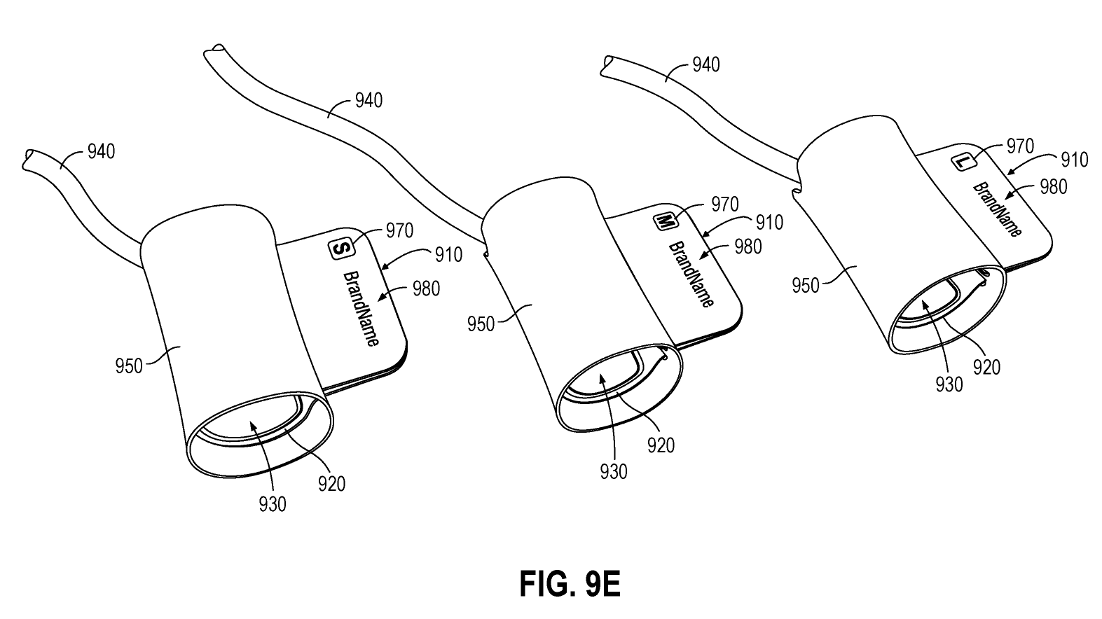

With additional reference to FIGS. 9A-9E, embodiments of the invention related to finger cuff 900 will be particularly described. In some embodiments, the finger cuff 900 may be the finger cuff 104, as previously described in FIG. 1. As shown, finger cuff 900 may include a fixed shell 950 and a wrapping portion 920 within the fixed shell 950. The fixed shell 950 may be of extended length and may be approximately cylindrically-shaped. It should be appreciated that the fixed shell 950 of the finger cuff 900 may be formed by any suitable sort of material: plastic, polyvinyl chloride (PVC), metallic material, etc., or combinations thereof, to provide suitable rigidity as well as desired flexibility.

Looking particularly at FIG. 9C, the top portion of the fixed shell 950 may include a first slot 965 and a second slot 967. Referring to FIGS. 9A-9D, for example, a first side 912 and second side 914 of the wrapping portion 920 may be inserted through first slot 965 and second slot 967, respectively, to form finger cavity 930 that is approximately oval shaped, and subsequently be fixed together on the interior to form a pull member 910. In one embodiment, the first side 912 and second side 914 may be permanently fixed together such that the wrapping portion 920 may not be removed from the fixed shell 950. In another embodiment, the first side 912 and second side 914 may be removably attached (e.g., using a Velcro clamping system) to each other such that the wrapping portion 920 may be removed from the fixed shell 950 and the wrapping portion 920 may particularly size the size of the finger cavity 930.

With continued reference to FIGS. 9A-9D, the pull member 910 may be pulled outward to tighten the wrapping portion 920 around the patient's finger. While applying the pull force on pull member 910, the pull member 910 may be turned clockwise (or counter-clockwise) to fix or secure to the exterior of the fixed shell 950. In one embodiment, the first side 912 or the second side 914 on the exterior may include a connecting portion (not shown), e.g., a velcro type portion, that connects with another connecting portion (also not shown), e.g., a velcro type portion, on the exterior of the fixed shell 950. In another embodiment, the exterior of the first side 912 or the second side 914 may include removable or reusable adhesive material that can be removably attached to the exterior of the fixed shell 950. It should be appreciated that these are just some examples of an attachment mechanism and that any suitable type may be utilized.

Looking at FIG. 9E in particular, the exterior of the first side 912 and/or the second side 914 may include a label 970 (e.g., a printed label) that displays information pertaining to the finger cuff 900. The label 970 may include a name brand 980, such as CLEARSIGHT from Edwards Lifesciences Corporation, and a letter or character or symbol portion 970 indicating the size of finger cuff 500. For example, the character portion 970 (which may be approximately square shaped) may include a letter “S” indicating a small-sized finger cuff 900, a letter “M” indicating a medium-sized finger cuff 900, and a letter “L” indicating a large-sized finger cuff 900. Other examples may include “XS” indicating extra small or “XL” indicating extra-large. Further, the character portion 970 may include different background colors (e.g., green, blue, and orange) to also indicate the size of finger cuff 900. For instance, a green background color may correlate to a small-sized finger cuff 900, a blue background color may correlate to a medium-sized finger cuff 900, and an orange background color may correlate to a large-sized finger cuff 900.

Although not explicitly shown in FIGS. 9A-9E, similar to the previously described embodiment, a bladder and an LED-PD pair (as previously described) may be mounted on the interior of the wrapping portion 920 (within the fixed shell 950) and within finger cavity 930. Further, the bladder and LED-PD pair may be coupled to tube or cable 940 through a connector (which may be any of connector and connector assembly as previously described, such as fixed connector 230, connector assembly 330, and snap connector assembly 420), with the tube 940 being guided through the fixed shell 950 via a shell cavity 960 (as shown in FIGS. 9B-9C) that receives the tube 940 at an edge of fixed shell 950. It should be appreciated that the bladder and the LED-PD pair may be used in measuring the patient's blood pressure by the blood pressure measurement system utilizing the volume clamping method.

It should be appreciated that aspects of the invention previously described may be implemented in conjunction with the execution of instructions by processors, circuitry, controllers, control circuitry, etc. As an example, control circuity may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff attachable to a patient's finger to be used in measuring the patient's blood pressure by a blood pressure measurement system, the finger cuff comprising:
a first side and a second side, the first side removably attached to the second side to wrap the finger cuff around the patient's finger;
a signal source and signal detector pair; and
a bladder including a pair of openings, the bladder mountable within the finger cuff such that the pair of openings surround the signal source and signal detector pair, respectively, wherein the patient's finger surrounded in the finger cuff abuts against the bladder such that the bladder and the signal source and signal detector pair are used in measuring the patient's blood pressure by the blood pressure measurement system, wherein the bladder and signal source and signal detector pair are coupled to a cable through a connector assembly, the cable providing pneumatic pressure to the bladder and receiving electrical signals from the signal source and signal detector pair.

2. The finger cuff of claim 1, wherein the signal source and signal detector pair include a light emitting diode (LED)—photodiode (PD) pair, respectively.

3. The finger cuff of claim 2, wherein the connector assembly comprises a plug connector removably attached to a receptacle connector, the plug connector including a first end connecting to the cable and a second end having a first plugging member and a second plugging member, wherein the receptacle connector includes a first end attached to the finger cuff and coupled to the bladder and LED-PD pair, and a second end having a first socket and a second socket such that the first plugging member is suitably pluggable into the second socket to receive electrical signals from the LED-PD pair, and the second plugging member is suitably pluggable into the first socket to provide pneumatic pressure to the bladder.

4. The finger cuff of claim 3, wherein the first plugging member of the plug connector comprises a group of electrical contacts that engage with a corresponding group of electrical contacts within the second socket of the receptacle connector when the first plugging member is plugged into the second socket, to provide power to the LED-PD pair and receive electrical signals from the LED-PD pair, and wherein the second plugging member of the plug connector comprises a cavity to pass pneumatic pressure through the first socket of the receptacle connector and to the bladder when the second plugging member is plugged into the first socket.

5. The finger cuff of claim 2, wherein the connector assembly comprises a snap member connected to the cable and having a loop extending from a surface of the snap member that is removably connected to a mating loop attached to an exterior of the finger cuff and coupled to the bladder and LED-PD pair.

6. The finger cuff of claim 2, further comprising a pair of sensor symbols indicating physical positions of the LED-PD pair within the finger cuff.

7. The finger cuff of claim 6, wherein the label and the pair of sensor symbols are located on an exterior of the finger cuff.

8. The finger cuff of claim 2, further comprising a label having a name brand and a character or symbol portion that displays a character indicative of size of the finger cuff.

9. The finger cuff of claim 8, wherein the character or symbol is any one of the following: XS, S, M, L, and XL, respectively, corresponding to extra small, small, medium, large, and extra-large.

10. A finger cuff connectable to a patient's finger to be used in measuring the patient's blood pressure by a blood pressure measurement system, the finger cuff comprising:
a top shell portion and a bottom shell portion forming a finger cavity of the finger cuff to be placed around the patient's finger, the bottom shell portion including a signal source and signal detector pair, and
a bladder including a pair of openings, the bladder mountable within the bottom shell portion such that the pair of openings surround the signal source and signal detector pair, respectively, wherein the patient's finger received and surrounded in the finger cavity abuts against the bladder such that the bladder and the signal source and signal detector pair are used in measuring the patient's blood pressure by the blood pressure measurement system.

11. The finger cuff of claim 10, wherein the signal source and signal detector pair include a light emitting diode (LED)—photodiode (PD) pair, respectively.

12. The finger cuff of claim 11, further comprising first and second opposing adjustable sidewalls extending from the top shell portion to the bottom shell portion, and a rotatable knob connected to an exterior of the top shell portion to adjust lengths of the first and second opposing adjustable sidewalls, thereby adjusting the size of the finger cavity surrounding the patient's finger.

13. The finger cuff of claim 12, wherein the lengths of the first and second sidewalls are shortened when the rotatable knob is turned clockwise to pull the bottom shell portion towards the top shell portion, thereby decreasing the size of the finger cavity, whereas the lengths of the first and second sidewalls are lengthened when the finger cuff handle is turned counter-clockwise to push the bottom shell portion away from the top shell portion, thereby increasing the size of the finger cavity.

14. The finger cuff of claim 12, wherein the bottom shell portion of the finger cuff is coupled to a cable through a connector assembly for providing pneumatic pressure to the bladder and receiving electrical signals from the LED-PD pair, the connector assembly comprising:
a plug connector including a first end connecting to the cable and a second end having a first plugging member and a second plugging member, and
a receptacle connector including a first end attached to the bottom shell portion and coupled to the bladder and LED-PD pair, and a second end having a first socket and a second socket such that the first plugging member is suitably pluggable into the second socket to receive electrical signals from the LED-PD pair, and the second plugging member is suitably pluggable into the first socket to provide pneumatic pressure to the bladder.

15. The finger cuff of claim 14, wherein the first plugging member of the plug connector comprises a group of electrical contacts that engage with a corresponding group of electrical contacts within the second socket of the receptacle connector when the first plugging member is plugged into the second socket, to provide power to the LED-PD pair and receive electrical signals from the LED-PD pair, and
wherein the second plugging member of the plug connector comprises a cavity to pass pneumatic pressure through the first socket of the receptacle connector and to the bladder when the second plugging member is plugged into the first socket.

16. A finger cuff connectable to a patient's finger to be used in measuring the patient's blood pressure by a blood pressure measurement system, the finger cuff comprising:
a shell having a first slot and a second slot on first and second opposing ends of the shell, the shell forming a finger cavity to be placed around the patient's finger;
a flexible material having a first side and a second side, wherein the first side and the second side are respectively inserted through the first slot and the second slot; and
a bladder assembly insertable into the shell, the bladder assembly comprising a shell portion having a signal source and signal detector pair, and a bladder including a pair of openings, the bladder mountable within the shell portion such that the pair of openings surround the signal source and signal detector pair, respectively, wherein the patient's finger received and surrounded in the finger cavity abuts against the bladder such that the bladder and the signal source and signal detector pair are used in measuring the patient's blood pressure by the blood pressure measurement system.

17. The finger cuff of claim 16, wherein the signal source and signal detector pair include a light emitting diode (LED)—photodiode (PD) pair, respectively.

18. The finger cuff of claim 17, wherein the bladder assembly further comprises a connector having one end connected to the shell portion and coupled to the bladder and LED-PD pair and another end connect to a cable, the cable providing pneumatic pressure through the connector to the bladder and receiving electrical signals from the LED-PD pair.

19. The finger cuff of claim 18, wherein the shell portion further includes a shell cavity at a convex edge of the shell portion, and wherein a portion of the connector is secured into the shell cavity when the bladder assembly is inserted into the shell.

20. The finger cuff of claim 16, wherein an interior of the first side of the flexible material is removably attached to a first outward-facing surface of the flexible material, and an interior of the second side of the flexible material is permanently attached to a second outward-facing surface of the flexible material.

21. The finger cuff of claim 20, wherein the first side of the flexible material is pullable to effectively tighten the flexible material around the patient's finger.

22. A finger cuff connectable to a patient's finger to be used in measuring the patient's blood pressure by a blood pressure measurement system, the finger cuff comprising:
a fixed shell including a first slot and a second slot; and
a wrapping portion within the fixed shell, the wrapping portion including a first side and a second side respectively inserted through the first slot and the second slot forming a finger cavity of the wrapping portion to be placed around the patient's finger, a signal source and signal detector pair, and a bladder including a pair of openings, the bladder mountable within the wrapping portion such that the pair of openings surround the signal source and signal detector pair, respectively, wherein, the patient's finger surrounded in the finger cavity abuts against the bladder such that the bladder and the signal source and signal detector pair are used in measuring the patient's blood pressure by the blood pressure measurement system.

23. The finger cuff of claim 22, wherein the signal source and signal detector pair include a light emitting diode (LED)—photodiode (PD) pair, respectively.

24. The finger cuff of claim 23, wherein interiors of the first side and second side of the wrapping portion are secured together forming a pull member pullable to effectively tighten the wrapping portion around the patient's finger, and wherein an exterior of the pull member is removably attached to an exterior of the fixed shell to effectively secure the patient's finger within the finger cavity.

25. The finger cuff of claim 24, wherein the fixed shell further includes a shell cavity that receives and guides a cable through the fixed shell, the cable coupling to the bladder and LED-PD pair, providing pneumatic pressure to the bladder and receiving electrical signals from the LED-PD pair.

26. The finger cuff of claim 24, wherein an exterior of the pull member comprises a label having a name brand and a character or symbol portion that displays a character indicative of size of the wrapping portion.

27. The finger cuff of claim 26, wherein the character or symbol is any one of the following: XS, S, M, L, and XL, respectively corresponding to extra small, small, medium, large, and extra-large.
